(19) United States
(12) Patent Application Publication
(10) Pub. No.: US 2019/0087908 A1
(43) Pub. Date: Mar. 21, 2019

(54) ELECTRONIC DEVICE, SERVER, AND PRIVILEGE INFORMATION PROVIDING METHOD AND PROGRAM

(71) Applicant: CASIO COMPUTER CO., LTD., Tokyo (JP)

(72) Inventor: Kazunori KITA, Tokyo (JP)

(73) Assignee: CASIO COMPUTER CO., LTD., Tokyo (JP)

(21) Appl. No.: 16/136,367

(22) Filed: Sep. 20, 2018

(30) Foreign Application Priority Data
Sep. 20, 2017 (JP) 2017-180436

(51) Int. Cl. G06Q 40/08; H04L 29/08

(52) U.S. Cl. CPC G06Q 40/08; G16H 50/30; H04L 67/22

(57) ABSTRACT

To more suitably provide beneficial information to a user, on the basis of information relevant to the regular life of the user. A wrist terminal includes a position acquisition unit, a sensor information acquisition unit, an activity information acquisition unit, and a privilege information providing unit. The position acquisition unit, the sensor information acquisition unit, and the activity information acquisition unit acquire factor information affecting a health condition of the user, on the basis of history information of the user relevant to at least one of date and time, a time (the length of time), and a location. The privilege information providing unit provides privilege information relevant to a risk requiring attention of the user, on the basis of the factor information acquired by the sensor information acquisition unit and the activity information acquisition unit.

### CROSS-REFERENCE TO RELATED APPLICATIONS

This application is based upon and claims the benefit of priority under 35 USC 119 of Japanese Patent Application No. 2017-180436 filed on Sep. 20, 2017 the entire disclosure of which, including the description, claims, drawings, and abstract, is incorporated herein by reference in its entirety.

### BACKGROUND OF THE INVENTION

### 1. Field of the Invention

The present invention relates to an electronic device, a server, and privilege information providing method and program.

### 2. Description of the Related Art

Conventionally, a technology is known in which a regular behavior of a user is determined, and a benefit is applied to the user on the basis of a determination result. For example, in JP 2005-141781 A, a technology is described in which information relevant to fitness, taking of a drug, ingestion of food, and the like of the user is acquired, an insurance expense suitable for the user is calculated on the basis of the acquired information.

### BRIEF SUMMARY OF THE INVENTION

One aspect of the present invention is An electronic device comprising: a memory; and a processor, wherein the processor executes a program stored in the memory to perform operations comprising: providing privilege information relevant to a risk requiring attention for a user, on the basis of history information of the user, wherein the history information is relevant to at least one of date and time, and a location.

### BRIEF DESCRIPTION OF THE SEVERAL VIEWS OF THE DRAWING

FIG. 1A is an external configuration diagram of a wrist terminal as one embodiment of an electronic device of the present invention FIG. 1B is a block diagram illustrating a hardware configuration of the wrist terminal as one embodiment of the electronic device of the present invention. FIG. 2 is a functional block diagram illustrating a functional configuration for executing privilege information providing processing, in functional configurations of the wrist terminal of FIGS. 1A and 1B. FIG. 3 is a schematic view illustrating an example of a conversion condition table. FIG. 4 is a flowchart illustrating a flow of the privilege information providing processing which is executed by the wrist terminal of FIGS. 1A and 1B including the functional configuration of FIG. 2. FIG. 5 is a schematic diagram illustrating a system configuration of a privilege information providing system according to a second embodiment of the present invention. FIG. 6 is a block diagram illustrating a hardware configuration of a privilege information providing server. FIG. 7 is a functional block diagram illustrating a functional configuration for executing terminal side privilege information providing processing, in functional configurations of a wrist terminal according to this embodiment. FIG. 8 is a functional block diagram illustrating a functional configuration for executing server side privilege information providing processing, in functional configurations of the privilege information providing server of FIG. 6. FIG. 9 is a flowchart illustrating a flow of the terminal side privilege information providing processing which is executed by the wrist terminal according to this embodiment. FIG. 10 is a flowchart illustrating a flow of the server side privilege information providing processing which is executed by the privilege information providing server.

### DETAILED DESCRIPTION OF THE INVENTION

Embodiments of the present invention will be explained with reference to the drawings.

Hereinafter, embodiments of the present invention will be described by using the drawings.

### First Embodiment

An electronic device according to this embodiment widely acquires information (hereinafter, referred to as “factor information”) affecting a health condition of a user (the health condition of the user, for example, is a physical or spiritual state of the user affected by trauma, spiritual stress, or disorder with respect to the user), from various life histories of the user. Furthermore, the life history includes various histories associated with the life of the user, such as movement history of the user (a movement in a transportation system, or the like), behavior history (a regular behavior or the like), activity history (a social activity or the like), fitness history (sports or the like), and environment history (a change of environment around the user, or the like). In this embodiment, the factor information is aggregated from activity data such as output of a sensor or a schedule input by the user himself/herself, and a regular behavior pattern, a special behavior, or the like of the user is grasped. In the factor information acquired as described above, a behavior that the user unconsciously performs (“seating time per day exceeds 10 hours in total” or the like), or a behavior that the user passively performed (“for example, staying in a specific area where a risk such as disaster or conflict, is high”, “a large temperature change in the surrounding environment”, or the like) is included, in addition to a behavior that the user consciously performs (“sports”, “meals”, or the like).

Then, the electronic device converts the factor information acquired by the user into information indicating various risks of the user, by a predetermined application program. That is, in the predetermined application, a method of calculating indices of influences of various factor information items on the various risks (here, referred to as a score), is defined. In the predetermined application, in a case where the factor information is input, the score of the influence of the factor information on the various risks is calculated, a noteworthy risk of the user is specified, and information for notifying the user (privilege information indicating advice) is output. Furthermore, information of encouraging a suitable behavior in a case where the user performs the behavior, can be included as the privilege information, in addition to the information of providing notification of the risk. That is, the privilege information functions as information of applying a benefit relevant to the factor information (benefit information), to the user. Accordingly, the factor information of the regular life of the user can be widely acquired, and beneficial information can be more suitably provided to the user by reflecting information relevant to the regular life of the user. Hereinafter, a specific embodiment will be described.

### [Hardware Configuration]

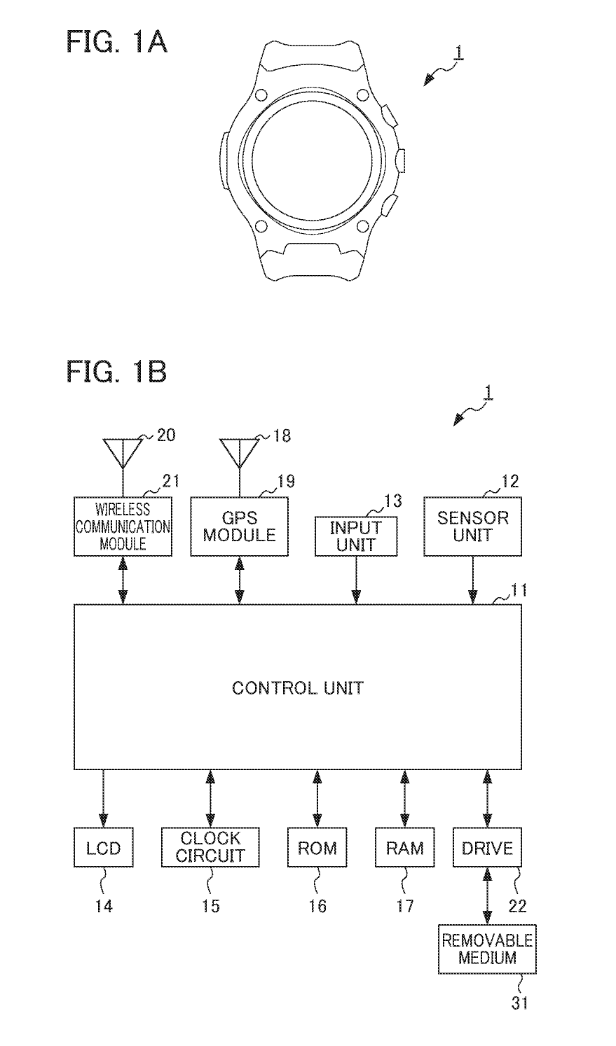

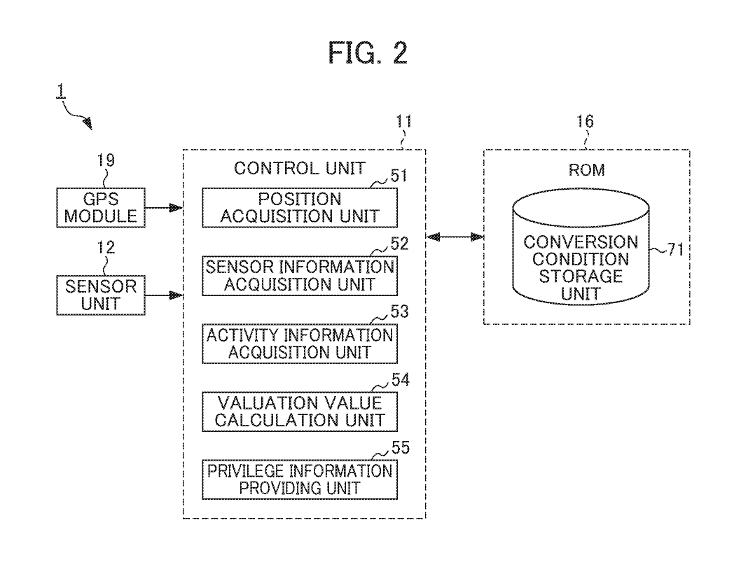

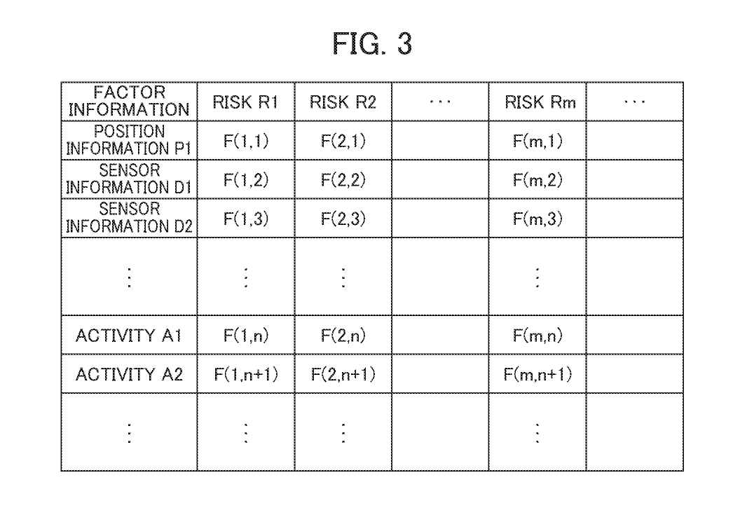

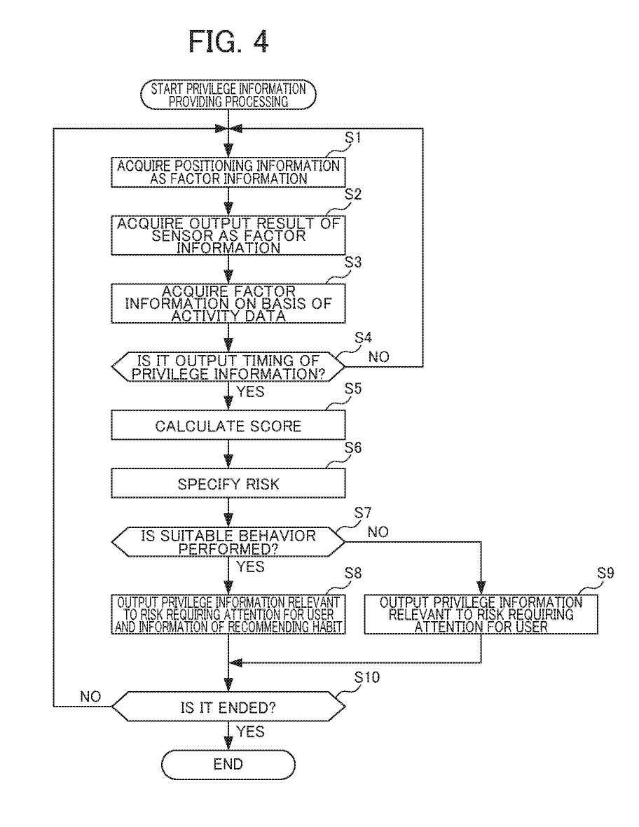

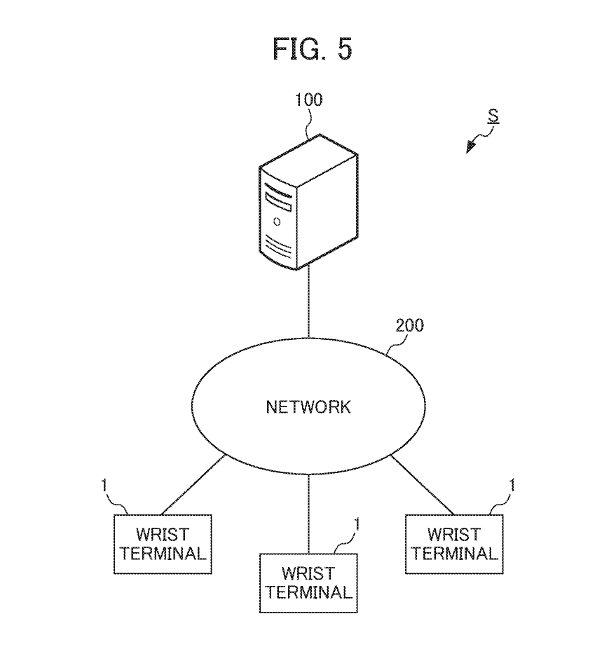

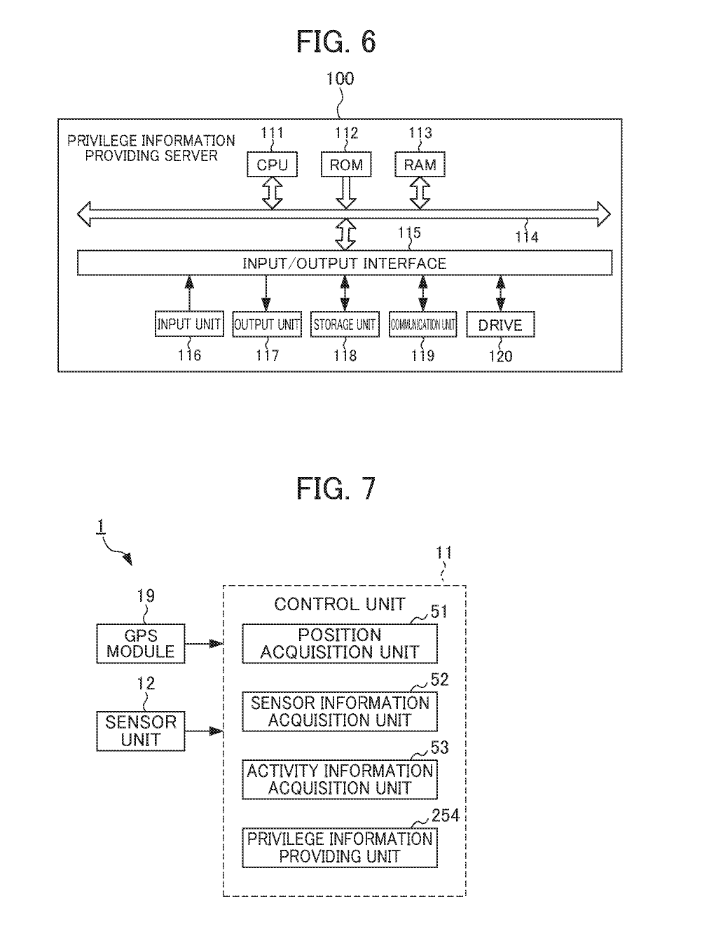

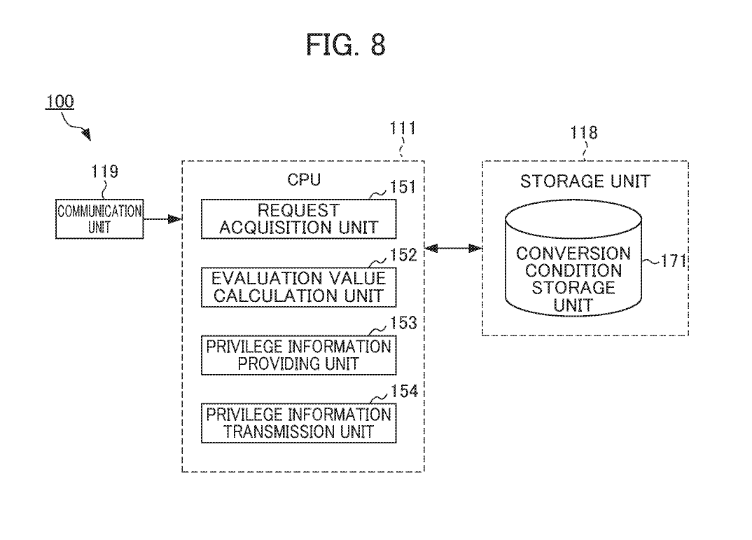

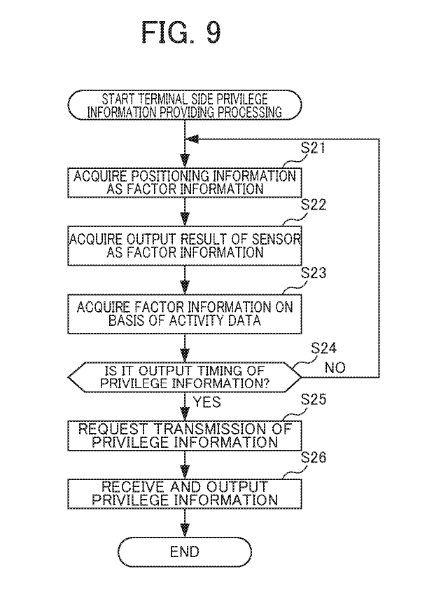

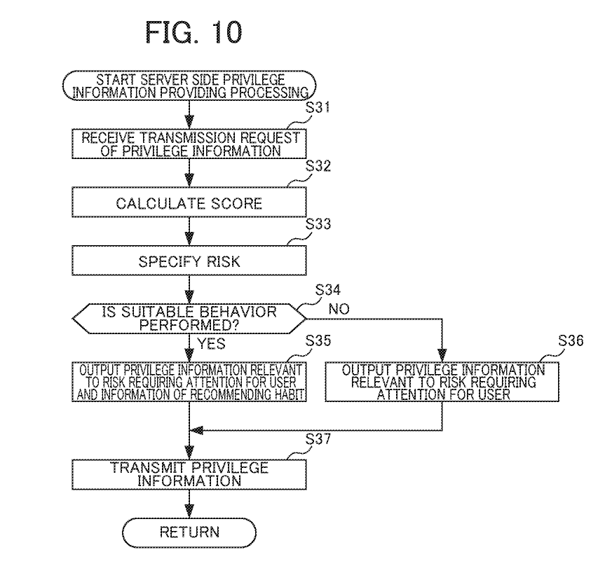

FIG. 1A and FIG. 1B are diagrams illustrating a configuration of a wrist terminal 1 as one embodiment of the electronic device of the present invention, FIG. 1A is an external configuration diagram, and FIG. 1B a block diagram illustrating a hardware configuration. As illustrated in FIG. 1A, the wrist terminal 1 is an electronic device which is configured in a wristwatch type, and has a function similar to a smart phone. In addition, as illustrated in FIG. 1B, the wrist terminal 1 includes a control unit 11, a sensor unit 12, an input unit 13, a liquid crystal display (LCD) 14, a clock circuit 15, a read only memory (ROM) 16, a read access memory (RAM) 17, a GPS antenna 18, a GPS module 19, a wireless communication antenna 20, a wireless communication module 21, and a drive 22.

The control unit 11 is configured of an arithmetic processing unit such as a central processing unit (CPU), and controls the operation of the entire wrist terminal 1. For example, the control unit 11 executes various processings according to a program recorded in the ROM 16, such as a program for privilege information providing processing (described below). The sensor unit 12 includes various sensors such as a biosensor (a pulse sensor, a blood pressure sensor, or the like), a geomagnetic sensor, a triaxial acceleration sensor, a triaxial angular velocity sensor, a humidity sensor, a barometric pressure sensor, or a temperature sensor.

The input unit 13 is configured of various buttons, an electrostatic capacitance type or resistance film type position input sensor laminated in a display region of the LCD 14, or the like, and inputs various information items according to an instruction operation of the user. The LCD 14 outputs an image according to an instruction of the control unit 11. For example, the LCD 14 displays a screen of various images or user interfaces. In this embodiment, the position input sensor of the input unit 13 is disposed to be superimposed on the LCD 14, and thus, a touch panel is configured. The clock circuit 15 generates a time signal from a signal which is generated by a system clock or an oscillator, and outputs the current time.

The ROM 16 is configured of a rewritable non-volatile semiconductor memory or the like, and stores information such as a control program which is executed by the control unit 11. For example, the RPM 16 stores the program for the privilege information providing processing described below. In this embodiment, the program for the privilege information providing processing is provided by an entity having regular authority for providing the privilege information. Therefore, the privilege information to be provided by executing the program for the privilege information providing processing, can be treated as having authenticity. The RAM 17 provides a work area at the time of executing various processings by the control unit 11. The GPS antenna 18 receives a radio wave which is transmitted from a satellite in a GPS, converts the received radio wave into an electric signal, and outputs the converted electric signal (hereinafter, referred to as a “GPS signal”) to the GPS module 19. The GPS module 19 detects the position of the wrist terminal 1 (a latitude, a longitude, and an altitude) and the current time indicated by the GPS, on the basis of the GPS signal input from the GPS antenna 18. In addition, the GPS module 19 outputs information indicating the detected position and current time to the control unit 11.

The wireless communication antenna 20 is an antenna which is capable of receiving a radio wave of a frequency corresponding to wireless communication, which is used by the wireless communication module 21, and for example, is configured of a loop antenna or a rod antenna. The wireless communication antenna 20 transmits an electric signal of wireless communication, which is input from the wireless communication module 21, as an electromagnetic wave, or outputs the received electromagnetic wave to the wireless communication module 21 by converting the electromagnetic wave into an electric signal.

The wireless communication module 21 transmits a signal to the other device through the wireless communication antenna 20, according to the instruction of the control unit 11. In addition, the wireless communication module 21 receives a signal transmitted from the other device, and outputs information indicated by the received signal, to the control unit 11. In this embodiment, the wireless communication module 21 performs communication based on a communication standard of Bluetooth low energy/Bluetooth LE (Trademark) (hereinafter, referred to as “BLE”), and communication based on a communication standard of wireless fidelity (Wi-Fi). In addition, the wireless communication module 21 is capable of performing communication with respect to the other wrist terminal 1 according to a beacon of the BLE. A removable medium 31, which is formed of a magnetic disk, an optical disk, a magnetooptical disk, or a semiconductor memory, or the like, is suitably mounted on the drive 22. The removable medium 31 is capable of storing various data items such as image data.

### [Functional Configuration]

Next, in functional configurations of the wrist terminal 1, a functional configuration for executing the privilege information providing processing will be described. FIG. 2 is a functional block diagram illustrating the functional configuration for executing the privilege information providing processing, in the functional configurations of the wrist terminal 1 of FIGS. 1A and 1B. The privilege information providing processing is a set of processings in which the various factor information items affecting the health condition of the user are acquired, the score indicating the influence of the acquired factor information on the various risks is calculated, the noteworthy risk of the user is specified, and the privilege information indicating the advice with respect to the user is output.

In a case where the privilege information providing processing is executed, as illustrated in FIG. 2, in the control unit 11, a position acquisition unit 51, a sensor information acquisition unit 52, an activity information acquisition unit 53, an evaluation value calculation unit 54, and a privilege information providing unit 55 function. In addition, a conversion condition storage unit 71 is disposed in one region of the ROM 16.

The conversion condition storage unit 71 stores a conversion condition table associated with a method of calculating the various factor information items, and the index (the score) of the influence of each of the factor information items on the various risks (a conversion condition for performing conversion from the factor information into the score). FIG. 3 is a schematic view illustrating an example of the conversion condition table. As illustrated in FIG. 3, in the conversion condition table, the method of calculating the influence of the factor information on the various risks (risks R1, R2 . . . ) (a function F(m,n) (m and n are natural numbers)) is individually defined with respect to the various factor information items (position information P1, sensor information items D1, D2 . . . , and activities A1, A2 . . . ).

For example, it is possible to calculate the score (an evaluation value) with respect to the factor information based on the positioning position, by multiplying a duration time during which a positioning position belongs to a specific position range (or a frequency in which the positioning position belongs to the specific position range, or the like), and a conversion factor set with respect to the positioning position for each type of risk, together. In addition, it is possible to calculate the score (the evaluation value) with respect to the factor information based on the output of the sensor, by multiplying an output value of the sensor, a duration time of the output value (or an occurrence frequency or the like), and a conversion factor set with respect to the output value of the sensor for each type of risk, together. In addition, it is possible to calculate the score with respect to the factor information based on the activity data, by multiplying a characteristic value according to the type of activity, and a conversion factor set with respect to the type of activity for each type of risk, together. In the characteristic value according to the type of activity, the characteristic value can be set with respect to the entire activity (a set of behaviors), or a value per unit time according to the type of activity (a reference value) can be set, and the duration time and the reference value can be multiplied together, and thus, the characteristic value can be obtained.

Furthermore, the conversion factor is a factor which is individually set according to the type of risk, for each output value of the sensor or each type of activity, and for example, a specific value can be set on the basis of statistical information obtained by analyzing an even that has actually occurred. In addition, in a case where the score is calculated, according to the type of factor information, the date and time of the occurrence of the factor information may be included in a calculation condition of the score. For example, in a case where there is the statistical data indicating that the risk of natural disaster such as river flooding increases in the specific area at a specific moment, when the movement history of the user corresponds to the specific area and the specific moment, the function F can be defined such that the score increases with respect to the risk of “the occurrence of disaster and an accident”.

Returning to FIG. 2, the position acquisition unit 51 acquires the positioning position of the wrist terminal 1 as the factor information. Specifically, the position acquisition unit 51 acquires the positioning position of the wrist terminal 1 as the factor information, on the basis of the position information acquired from the GPS module 19. Furthermore, the position acquisition unit 51 accumulates and stores the positioning position of the wrist terminal 1 (the factor information) in the ROM 16. The sensor information acquisition unit 52 acquires an output result of various sensors provided in the sensor unit 12, as the factor information. For example, the sensor information acquisition unit 52 acquires biological information of the user of the wrist terminal 1, which is output by a biosensor, or fitness information of the user of the wrist terminal 1, which is output by a triaxial acceleration sensor or the like, as the factor information. Furthermore, the sensor information acquisition unit 52 accumulates and stores the acquired output result of the various sensors (the factor information) in the ROM 16.

The activity information acquisition unit 53 acquires the activity data such as the schedule input by the user himself/herself. For example, the activity information acquisition unit 53 acquires various activity data items that the user performs, such as the desk work of the user in the office, frequent entrance and exit with respect to a building, exposure with respect to direct sunlight for a long period of time, and participation in a regional activity, with reference to the data of the schedule of the user. At this time, the activity information acquisition unit 53 may perform behavior analysis of the user, and may determine an activity that the user is performing. Then, the activity information acquisition unit 53 acquires the factor information affecting the health condition of the user, on the basis of the acquired activity data. At this time, the activity information acquisition unit 53 acquires not only one factor information item from the data such as one schedule of the user, but also factor information in a case where the same schedule is captured from a plurality of viewpoints. For example, in a case where a schedule of “staying an area X for one week on a business trip” is set, the activity information acquisition unit 53 acquires factor information of “a movement to the area X in a transportation system”, “continuously eating outside for one week”, and “the influence of regional characteristics of the area X for one week”. Furthermore, in a case where the area X is an area in which the risk of a high blood pressure is statistically high, the factor information of “the influence of the regional characteristics of the area X for one week” acts to increase the score with respect to the risk of the high blood pressure. Furthermore, the activity information acquisition unit 53 accumulates and stores the factor information affecting the health condition of the user, which is acquired on the basis of the acquired activity data, in the ROM 16.

The evaluation value calculation unit 54 refers to the conversion condition table, on the basis of the factor information acquired by the position acquisition unit 51, the sensor information acquisition unit 52, and the activity information acquisition unit 53, and calculates the score indicating the influences of the various factor information items on the various risks.

The privilege information providing unit 55 determines the level of the risk of the user, on the basis of the score calculated by the evaluation value calculation unit 54, with respect to the various risks, and specifies the noteworthy risk of the user. Then, the privilege information providing unit 55 outputs the privilege information for notifying the user of the specified risk, to the LCD 14. In a case where the level of the risk of the user is determined, the privilege information providing unit 55 compares the total value of the score of the user, which is calculated with respect to the various risks, with a threshold value set in advance, and determines whether or not the total value of the score of the user exceeds the threshold value, as an example. The total value of the score of the user may be suitably normalized and calculated according to the type of acquired factor information, or the like. In addition, the privilege information providing unit 55 is capable of including advice for reducing the risk, as the privilege information. For example, the privilege information providing unit 55 is capable of including advice for suppressing a factor contributing much to an increase in the score in the factor information (a message of “The seating time seems to be too long. Let's stand up once every 30 minutes in order to reduce the risk of backache.”, or the like).

In addition, the privilege information providing unit 55 determines whether or not the user performs a suitable behavior, on the basis of the score which is calculated by the evaluation value calculation unit 54, with respect to the various risks. Then, in a case where it is determined that the user performs the suitable behavior, on the basis of the score which is calculated by the evaluation value calculation unit 54, with respect to the specific risk, the privilege information providing unit 55 outputs information of encouraging the behavior by including the information in the privilege information. In a case where the fitness history in the regular life of the user contributes to health (appropriate for a specific condition for health), the privilege information providing unit 55 outputs information of recommending fitness habit of the user, as the privilege information, as an example. At this time, in a case where the application program for the privilege information providing processing is provided from an insurance company, information on substantial privilege of “Since the fitness habit is appropriate for a condition contributing to the health, the next insurance expense will be 10% discount!”, can be provided, as the privilege information to be provided to the user from the insurance company.

### [Operation]

Next, the operation of the wrist terminal 1 will be described. FIG. 4 is a flowchart illustrating a flow of the privilege information providing processing which is executed by the wrist terminal 1 of FIGS. 1A and 1B, having the functional configuration of FIG. 2. The privilege information providing processing is started by a privilege information providing processing start operation of the user with respect to the input unit 13.

In step S1, the position acquisition unit 51 acquires the positioning position of the wrist terminal 1 as the factor information, on the basis of the position information acquired from the GPS module 19. In step S2, the sensor information acquisition unit 52 acquires the output result of the various sensors provided in the sensor unit 12, as the factor information. In step S3, the activity information acquisition unit 53 acquires the activity data such as the schedule input by the user himself/herself, and acquires the factor information affecting the health condition of the user, on the basis of the acquired activity data.

In step S4, the evaluation value calculation unit 54 determines whether or not it is an output timing of the privilege information. Furthermore, whether or not it is the output timing of the privilege information, for example, can be determined by whether or not a period set in advance (one day, one week, one month, or the like) has elapsed, or whether or not an instruction is given on the output of the privilege information by the user. In a case where it is the output timing of the privilege information, it is determined as YES in step S4, and the processing proceeds to step S5. On the other hand, in a case where it is not the output timing of the privilege information, it is determined as NO in step S4, and the processing proceeds to step S1.

In step S5, the evaluation value calculation unit 54 refers to the conversion condition table, on the basis of the factor information acquired by the position acquisition unit 51, the sensor information acquisition unit 52, and the activity information acquisition unit 53, and calculates the score indicating the influence of the various factor information items on the various risks. In step S6, the privilege information providing unit 55 determines the level of the risk of the user, on the basis of the score which is calculated by the evaluation value calculation unit 54, with respect to the various risks, and the noteworthy risk of the user is specified. In step S7, the privilege information providing unit 55 determines whether or not the user performs the suitable behavior, on the basis of the score which is calculated by the evaluation value calculation unit 54, with respect to the various risks.

In a case where the user performs the suitable behavior, it is determined as YES in step S7, and the processing proceeds to step S8. On the other hand, in a case where the user does not perform the suitable behavior, it is determined as NO in step S7, and the processing proceeds to step S9. In step S8, the privilege information providing unit 55 outputs the privilege information relevant to the specified risk requiring attention for the user and information of recommending the habit of the user, to the LCD 14. After step S8, the processing proceeds to step S10. In step S9, the privilege information providing unit 55 outputs the privilege information relevant to the specified risk requiring attention for the user, to the LCD 14.

In step S10, the privilege information providing unit 55 determines whether or not an instruction is given on the end of the privilege information providing processing. In a case where the instruction is not given on the end of the privilege information providing processing, it is determined as NO in step S10, and the processing proceeds to step S1. Do the other hand, in a case where the instruction is given on the end of the privilege information providing processing, it is determined as YES in step S10, and the privilege information providing processing is ended.

As described above, the wrist terminal 1 according to this embodiment widely acquires the factor information, on the basis of the positioning position or the output of the sensor, or the activity data such as the schedule input by the user himself/herself. Then, the wrist terminal 1 converts the factor information acquired with respect to the user into the information indicating the various risks of the user, by the predetermined application program. Further, the wrist terminal 1 calculates the score of the influence of the factor information on the various risks, specifies the noteworthy risk of the user, and outputs the information for notifying the user (the privilege information indicating the advice). At this time, in a case where the user performs the suitable behavior, the wrist terminal 1 outputs the information of recommending the behavior by including the information in the privilege information. In the conventional technology, information which is acquired as the regular behavior of the user is limited, and it is not easy to acquire such information. In addition, there is a case where it is not sufficient to apply a benefit to the user, and then, only to determine the regular behavior of the user, as a determination element. In contrast, the wrist terminal 1 according to this embodiment, the factor information in the regular life of the user can be widely acquired, and the beneficial information can be more suitably provided to the user by reflecting the information relevant to the regular life of the user.

For example, in a case where there is the statistical data indicating that the risk of the natural disaster such as the river flooding, increases in the specific area at the specific moment, and the movement history of the user corresponds to the specific area and the specific moment, the wrist terminal 1 is capable of outputting an increase in the risk of the occurrence of the disaster or the accident, and the advice for suppressing the factor information (a message of “In XX area, currently, the risk of the natural disaster is high. Do not approach XX area as much as possible.”, or the like), as the privilege information.

In addition, for example, in a case where a change in the barometric pressure which is grasped from the sensor information, extremely increases according to frequent entrance and exit of the user with respect to a highly airtight building, frequent use of the elevator, or the like (in the case of exceeding the set threshold value of the score), the wrist terminal 1 is capable of outputting a large fluctuation in the barometric pressure of the surrounding environment, and an increase in a risk such as headache or joint ache, and the advice for suppressing the factor information (a message of “Be careful with a behavior causing a large change in the barometric pressure, such as entrance and exit with respect to a highly airtight building or room.”, or the like), as the privilege information.

In addition, for example, in a case where the number of days in which the sum of the fitness that the user performs for one day, is longer than or equal to 60 minutes, is greater than 20 consecutive days in this month, the wrist terminal 1 is capable of outputting the suitableness of the current fitness situation, and the information on the substantial privilege (the message of “Since the fitness habit is appropriate for a condition contributing to the health, the next insurance expense will be 10% discount!”, or the like), as the privilege information. Furthermore, the total time of the fitness that the user performs for one day, can be calculated by also accumulating a fitness time that the user is not conscious, such as going up and down the stairs, walking in the building, or running for a short time, in the factor information acquired by the wrist terminal 1.

### Second Embodiment

Next, a second embodiment of the present invention will be described. In this embodiment, the main processing performed by the evaluation value calculation unit 54 and the privilege information providing unit 55 in the first embodiment, is performed by a predetermined server.

### [System Configuration]

FIG. 5 is a schematic view illustrating a system configuration of a privilege information providing system S according to the second embodiment of the present invention. As illustrated in FIG. 5, the privilege information providing system S is configured by including the wrist terminal 1 and a privilege information providing server 100, and the wrist terminal 1 and the privilege information providing server 100 are configured to communicate with each other through a network 200.

### [Hardware Configuration]

In FIG. 5, a hardware configuration of the wrist terminal 1 is identical to the configuration illustrated in FIG. 1A and FIG. 1B of the first embodiment. FIG. 6 is a block diagram illustrating a hardware configuration of the privilege information providing server 100. As illustrated in FIG. 6, the privilege information providing server 100 includes a CPU 111, a ROM 112, a RAM 113, a bus 114, an input/output interface 115, an input unit 116, an output unit 117, a storage unit 118, a communication unit 119, and a drive 120. The CPU 111 executes various processings according to a program which is recorded in the ROM 112, or a program which is loaded in the RAM 113 from the storage unit 118. Data necessary for the CPU 111 to execute the various processings, is also suitably stored in the RAM 113.

The CPU 111, the ROM 112, and the RAM 113 are connected to each other through the bus 114. The bus 114 is connected to the input/output interface 115. The input unit 116, the output unit 117, the storage unit 118, the communication unit 119, and the drive 120 are connected to the input/output interface 115.

The input unit 116 is configured of various buttons or the like, and inputs various information items according to the instruction operation of the user. The output unit 117 is configured of a display, a speaker, or the like, and outputs an image or a sound. The storage unit 118 is configured of a hard disk or a dynamic random access memory (DRAM), or the like, and stores various data items. The communication unit 119 controls wireless or wired communication which is performed with respect to the other device through a network including the Internet.

A removable medium (not illustrated), formed of a magnetic disk, an optical disk, a magnetooptical disk, a semiconductor memory, or the like, is suitably mounted on the drive 120. A program which is read out from the removable medium by the drive 120, is installed in the storage unit 118 as necessary. In addition, the removable medium is capable of also storing the various data items stored in the storage unit 118, as with the storage unit 118.

### [Functional Configuration]

Next, a functional configuration of the privilege information providing system S will be described. Initially, a functional configuration of the wrist terminal 1 will be described. FIG. 7 is a functional block diagram illustrating a functional configuration for executing terminal side privilege information providing processing, in functional configurations of the wrist terminal 1 according to this embodiment. The terminal side privilege information providing processing is a set of processings in which the various factor information items affecting the health condition of the user are acquired, the acquired factor information is transmitted to the privilege information providing server 100 along with the position information of the wrist terminal 1, and thus, transmission of the privilege information indicating the advice with respect to the user is requested to the privilege information providing server 100, and the privilege information transmitted from the privilege information providing server 100 is output.

In a case where the terminal side privilege information providing processing is executed, as illustrated in FIG. 8, in the control unit 11, the position acquisition unit 51, the sensor information acquisition unit 52, the activity information acquisition unit 53, and a privilege information providing unit 254 function. Among them, functional configurations of the position acquisition unit 51, the sensor information acquisition unit 52, and the activity information acquisition unit 53 are identical to the configurations of each unit illustrated in FIG. 2. The privilege information providing unit 254 transmits the factor information which is acquired by the position acquisition unit 51, the sensor information acquisition unit 52, and the activity information acquisition unit 53, to the privilege information providing server 100, and thus, requests the transmission of the privilege information indicating the advice with respect to the user. Then, the privilege information providing unit 254 receives and outputs the privilege information transmitted from the privilege information providing server 100.

Next, a functional configuration of the privilege information providing server 100 will be described. FIG. 8 is a functional block diagram illustrating a functional configuration for executing server side privilege information providing processing, in functional configurations of the privilege information providing server 100 of FIG. 6. The server side privilege information providing processing is a set of processings in which in a case where the request of the transmission of the privilege information is performed from the wrist terminal 1, the noteworthy risk of the user is specified, on the basis of the factor information acquired by the position acquisition unit 51, the sensor information acquisition unit 52, and the activity information acquisition unit 53, and the privilege information for notifying the user of the specified risk is transmitted.

In a case where the server side privilege information providing processing is executed, as illustrated in FIG. 9, in the CPU 111, a request acquisition unit 151, an evaluation value calculation unit 152, a privilege information providing unit 153, and a privilege information transmission unit 154 function. In addition, a conversion condition storage unit 171 is disposed in one region of the storage unit 118. Among them, the configuration of the conversion condition storage unit 171 is identical to the configuration of the conversion condition storage unit 71 in FIG. 2.

The request acquisition unit 151 receives a transmission request of the privilege information, along with the various factor information items, from the wrist terminal 1. Furthermore, the various factor information items of the wrist terminal 1 are accumulated data during a predetermined period in the wrist terminal 1. The evaluation value calculation unit 152 refers to the conversion condition table, on the basis of the various factor information items transmitted from the wrist terminal 1, and calculates the score indicating the influence of the various factor information items on the various risks.

The privilege information providing unit 153 determines the level of the risk of the user, on the basis of the score which is calculated by the evaluation value calculation unit 152, with respect to the various risks, and specifies the noteworthy risk of the user. Then, the privilege information providing unit 153 outputs the privilege information for notifying the user of the specified risk, to the privilege information transmission unit 154. In a case where the level of the risk of the user is determined, the privilege information providing unit 55 compares the total value of the score of the user, which is calculated with respect to the various risks, with the threshold value set in advance, and determines whether or not the total value of the score of the user exceeds the threshold value, as an example. The total value of the score or the user may be suitably normalized and calculated according to the type of factor information to be acquired, or the like. In addition, the privilege information providing unit 153 is capable of including the advice for reducing the risk, as the privilege information. For example, the privilege information providing unit 153 is capable of including the advice for suppressing the factor contributing much to the increase in the score in the factor information (the message of “The seating time seems to be too long. Let's stand up once every 30 minutes in order to reduce the risk of backache.”, or the like).

In addition, the privilege information providing unit 153 determines whether or not the user performs the suitable behavior, on the basis or the score which is calculated by the evaluation value calculation unit 152, with respect to the various risks. Then, in a case where it is determined that the user performs the suitable behavior with respect to the specific risk, on the basis of the score which is calculated by the evaluation value calculation unit 152, the privilege information providing unit 153 outputs the information of recommending the behavior by including the information in the privilege information. In a case where the fitness history in the regular life of the user contributes to the health, the privilege information providing unit 153 outputs the information of recommending the fitness habit of the user, as the privilege information, as an example. At this tame, in a case where the application program for the privilege information providing processing is provided from the insurance company, the information on the substantial privilege of “Since the fitness habit is appropriate for the condition contributing to the health, the next insurance expense will be 10% discount!”, can be provided, as the privilege information which is provided to the user from the insurance company. The privilege information transmission unit 154 transmits the privilege information output by the privilege information providing unit 153, to the wrist terminal 1.

### [Operation]

Next, the operation of the privilege information providing system S will be described. Initially, the operation of the wrist terminal 1 will be described. FIG. 9 is a flowchart illustrating a flow of the terminal side privilege information providing processing which is executed by the wrist terminal 1 according to this embodiment. The terminal side privilege information providing processing is started by a terminal side privilege information providing processing start operation of the user with respect to the input unit 13.

In step S21, the position acquisition unit 51 acquires the positioning position of the wrist terminal as the factor information, on the basis of the position information acquired from the GPS module 19. In step S22, the sensor information acquisition unit 52 acquires the output result of the various sensors provided in the sensor unit 12, as the factor information. In step S23, the activity information acquisition unit 53 acquires the activity data such as the schedule input by the user himself/herself, and acquires the factor information affecting the health condition of the user, on the basis of the acquired activity data.

In step S24, the privilege information providing unit 254 determines whether or not it is the output timing of the privilege information. Furthermore, whether or not it is the output timing of the privilege information, for example, can be determined by whether or not the period set in advance (one day, one week, one month, or the like) has elapsed, or whether or not an instruction is given on the output of the privilege information by the user. In a case where it is the output timing of the privilege information, it is determined as YES in step S24, and the processing proceeds to step S25. On the other hand, in a case where it is not the output timing of the privilege information, it is determined as NO in step S24, and the processing proceeds to step S21.

In step S25, the factor information acquired by the position acquisition unit 51, the sensor information acquisition unit 52, and the activity information acquisition unit 53, is transmitted to the privilege information providing server 100, and thus, the transmission of the privilege information indicating the advice with respect to the user is requested. In step S26, the privilege information providing unit 254 receives and outputs the privilege information which is transmitted from the privilege information providing server 100. After step S26, the terminal side privilege information providing processing is ended.

Next, the operation of the privilege information providing server will be described. FIG. 10 is a flowchart illustrating a flow of the server side privilege information providing processing which is executed by the privilege information providing server 100. The server side privilege information providing processing is started along with the activation of the privilege information providing server 100, and is repeatedly executed. In step 331, the request acquisition unit 151 receives the transmission request of the privilege information, along with the various factor information items, from the wrist terminal 1.

In step S32, the evaluation value calculation unit 152 refers to the conversion condition table, on the basis of the various factor information items transmitted from the wrist terminal 1, and calculates the score indicating the influence of the various factor information items on the various risks. In step S33, the privilege information providing unit 153 determines the level of the risk of the user, on the basis of the score which is calculated by the evaluation value calculation unit 152, with respect to the various risks, and specifies the noteworthy risk of the user.

In step S34, the privilege information providing unit 153 determines whether or not the user performs the suitable behavior, on the basis or the score which is calculated with the evaluation value calculation unit 152, with respect to the various risks. In a case where the user performs the suitable behavior, it is determined as YES in step S34, and the processing proceeds to step S35. On the other hand, in a case where the user does not perform the suitable behavior, it is determined as NO in step S34, and the processing proceeds to step S36.

In step S35, the privilege information providing unit 153 outputs the privilege information relevant to the specified risk requiring attention for the user and the information of recommending the habit of the user, to the privilege information transmission unit 154. After step S35, the processing proceeds to step S37. In step S36, the privilege information providing unit 153 outputs the privilege information relevant to the specified risk requiring attention for the user, to the privilege information transmission unit 154. In step S37, the privilege information transmission unit 154 transmits the privilege information output by the privilege information providing unit 153, to the wrist terminal 1. After step S37, the server side privilege information providing processing is repeated.

As described above, in the privilege information providing system S according to this embodiment, the privilege information providing server 100 specifies the noteworthy risk of the user, on the basis of the various factor information items transmitted from the wrist terminal 1, and transmits the privilege information for notifying the user. Therefore, by reflecting the information relevant to the regular life of the user, which is acquired in the wrist terminal 1, it is possible to more suitably provide the beneficial information to the user from the privilege information providing server 100.

The wrist terminal 1 configured as described above, includes the position acquisition unit 51, the sensor information acquisition unit 52, the activity information acquisition unit 53, and the privilege information providing unit 55. The position acquisition unit 51, the sensor information acquisition unit 52, and the activity information acquisition unit 53 acquire the factor information affecting the health condition of the user, on the basis of the history information of the user, wherein the history information is relevant to at least one of the date and time, a time (the length of time), and a location. The privilege information providing unit 55 provides the privilege information relevant to a risk requiring attention of the user, on the basis of the factor information which is acquired by the sensor information acquisition unit 52 and the activity information acquisition unit 53. Accordingly, it is possible to widely acquire the factor information in the regular life of the user, and to more suitably provide the beneficial information to the user, by reflecting the information relevant to the regular life of the user.

In addition, the wrist terminal 1 includes the evaluation value calculation unit 54. The evaluation value calculation unit 54 calculates the evaluation value indicating the influence of the factor information on the risk, which is acquired by the position acquisition unit 51, the sensor information acquisition unit 52, and the activity information acquisition unit 53, for each type of risk. The privilege information providing unit 55 determines the level of the risk for each type of risk, on the basis of the evaluation value calculated by the evaluation value calculation unit 54, and specifies the risk requiring attention of the user. Accordingly, it is possible to determine the level of the risk by suitably reflecting the factor information for each type of risk, and to more accurately grasp the various risks from the acquired factor information.

The privilege information providing unit 55 specifies the risk requiring attention of the user by reflecting each influence of at least two of the date and time, the time, and the location in the factor information, on the risk. Accordingly, it is possible to specify the risk requiring attention of the user by accurately reflecting individual influence of each of the date and time, the time, and the location, wherein they are relevant to the factor information, on the risk.

The factor information includes factor information depending on an area where the user stays. Accordingly, in a case where there is a regional difference in the occurrence of the risk, or in a case where the specific risk occurs in the specific area, it is possible to suitably specify a risk having a possibility of indirectly affecting the user.

The factor information includes factor information depending on the environment around the user. Accordingly, in a case where a risk occurs due to an environmental factor other than an independent factor of the user, it is possible to suitably specify the risk having a possibility of indirectly affecting the user.

The privilege information providing unit 55 provides the privilege information including advice information for suppressing the factor information, with respect to the risk requiring attention of the user. Accordingly, it is possible to provide accurate advice information for reducing the risk requiring attention of the user.

The privilege information providing unit 55 determines whether or not the history information of the user is appropriate for the specific condition determined as suitable with respect to the health, on the basis of the factor information acquired by the position acquisition unit 51, the sensor information acquisition unit 52, and the activity information acquisition unit 53, and in a case where the history information of the user is appropriate for the specific condition, privilege information for applying a benefit relevant to the insurance is provided to the user. Accordingly, it is possible to encourage the user to perform the suitable behavior for the health. In addition, the motivation for acquiring the factor information increases for many users, and thus, it is possible for the insurance company or the like to easily acquire the factor information of many users.

In addition, the privilege information providing system S includes the wrist terminal 1 and the privilege information providing server 100. The privilege information providing server 100 includes the request acquisition unit 151, the privilege information providing unit 153, and the privilege information transmission unit 154. The request acquisition unit 151 acquires a privilege information providing request including the factor information affecting the health condition of the user, based on the history information of the user, wherein the history information is relevant to at least one of the date and time, the time, and the location, from the wrist terminal 1. The privilege information providing unit 153 acquires the privilege information relevant to the risk requiring attention of the user, on the basis of the factor information acquired by the request acquisition unit 151. The privilege information transmission unit 154 transmits the privilege information acquired by the privilege information providing unit 153, to the wrist terminal 1. Accordingly, it is possible to widely acquire the factor information in the regular life of the user, and to more suitably provide the beneficial information of the user, by reflecting the information relevant to the regular life of the user.

It should be noted that the present invention is riot to be limited to the aforementioned embodiments, and that modifications, improvements, etc. within a scope that can achieve the objects of the present invention are also included in the present invention.

In the embodiments described above, the noteworthy risk of the user, the advice information for reducing the risk, or the privilege information to be provided to the user from the insurance company (information relevant to a discount of the insurance expense, or the like) is output as the privilege information, but the privilege information is not limited thereto. For example, information for providing a benefit such as a coupon, a gift, or electronic money to the user may be output as the privilege information.

In addition, in the embodiments described above, the privilege information is output as text data such as a message, but is not limited thereto. For example, the risk specified from the factor information can be displayed on a map, or can be displayed on a graph indicating a chronological change. In addition, in the embodiments described above, the risk relevant to the natural disaster has been described as the risk in the specific area, but is not limited thereto. For example, a risk related to artificial disaster such as the level of an accident rate, environmental pollution, and natural destruction, may be considered. In addition, in the embodiments described above, the score to be calculated in order to specify the risk, can be calculated by various methods, and for example, can be calculated by the following calculation methods.
(1) A multiplication type “Score=Occurrence Probability×Level of Influence” or “Score=Exposure Amount (Occurrence Probability)×Toxicity (Influence Rate)”
(2) An addition type “Score=Occurrence Probability (Score)+Level of Influence (Score)”
(3) A scatter diagram type “Score=Position Relationship when Occurrence Probability and Level of Influence are Plotted as Scatter Diagram”
In addition, in the embodiments described above, the factor information is acquired from various life histories of the user, and the score of the influence on the various risks of the user is calculated by the predetermined application program, the noteworthy risk of the user is specified, and the privilege information is output, as the factor information, and as another configuration, not only is the influence on the various risks of the user calculated from the factor information, but also a list is provided in which the various factor information items are associated with each influence on the various risks of the user, and a list is provided in which each influence on the various risks of the user is associated with various privilege information items. Then, in a case where the factor information is acquired, the information relevant to the influence on the risk or the privilege information may be acquired by referring to the list. In addition, in the embodiments described above, the factor information is acquired from the various life histories of the user, the score of the influence on the various risks of the user is calculated by the predetermined application program, the noteworthy risk of the user is specified, and the privilege information is output, as the factor information, and as another configuration, in a case where the various life histories of the user are acquired, the privilege information may be directly acquired from the information. At this time, a list may be prepared in which various life history information items of various users are associated with the various privilege information items, and the privilege information based on the life history information may be acquired by using the list, or an index value may be calculated by using a score set for each life history information item of the user, and the privilege information based on the life history information may be acquired on the basis of the index value.

In addition, in the embodiments described above, the wristwatch type electronic device similar to a smart phone has been described as the wrist terminal 1 which is applied to the present invention, but the wrist terminal 1 is not particularly limited. For example, the present invention can be applied to a general electronic device having an information processing function. Specifically, for example, the present invention can be applied to a notebook personal computer, a video camera, a portable navigation device, a mobile phone, a smart phone, a portable game machine, or the like.

The processing sequence described above can be executed by hardware, and can also be executed by software. In other words, the hardware configuration of FIGS. 2, 7 and 8 is merely illustrative examples, and the present invention is not particularly limited thereto. More specifically, the types of functional blocks employed to realize the above-described functions are not particularly limited to the examples shown in FIGS. 2, 7 and 8, so long as the wrist terminal 1 or the privilege information providing server 100 can be provided with the functions enabling the aforementioned processing sequence to be executed in its entirety. A single functional block may be constituted by a single piece of hardware, a single installation of software, or a combination thereof.

The functional configurations of the present embodiment are realized by a processor executing arithmetic processing, and processors that can be used for the present embodiment include a unit configured by a single unit of a variety of single processing devices such as a single processor, multi-processor, multi-core processor, etc., and a unit in which the variety of processing devices are combined with a processing circuit such as ASIC (Application Specific integrated Circuit) or FPGA (Field-Programmable Gate Array).

In the case of having the series of processing executed by software, the program constituting this software is installed from a network or storage medium to a computer or the like. The computer may be a computer equipped with dedicated hardware. In addition, the computer may be a computer capable of executing various functions, e.g., a general purpose personal computer, by installing various programs.

The storage medium containing such a program can not only be constituted by the removable medium 31 of FIG. 1 distributed separately from the device main body for supplying the program to a user, but also can be constituted by a storage medium or the like supplied to the user in a state incorporated in the device main body in advance. The removable medium 31 is composed of, for example, a magnetic disk (including a floppy disk), an optical disk, a magnetic optical disk, or the like. The optical disk is composed of, for example, a CD-ROM (Compact Disk-Read Only Memory), a DVD (Digital Versatile Disk), Blu-ray (Registered Trademark) or the like. The magnetic optical disk is composed of an MD (Mini-Disk) or the like. The storage medium supplied to the user in a state incorporated in the device main body in advance is constituted by, for example, the ROM 12 of FIG. 1 in which the program is recorded, and a hard disk included in the storage unit 20 of FIG. 1, and the like.

It should be noted that, in the present specification, the steps defining the program recorded in the storage medium include not only the processing executed in a time series following this order, but also processing executed in parallel or individually, which is not necessarily executed in a time series. Further, in this specification, the term of the system shall mean an entire apparatus composed of a plurality of apparatuses, a plurality of means and the like.

The embodiments of the present invention described above are only illustrative, and are not to limit the technical scope of the present invention. The present invention can assume various other embodiments. Additionally, it is possible to make various modifications thereto such as omissions or replacements within a scope not departing from the spirit of the present invention. These embodiments or modifications thereof are within the scope and the spirit of the invention described in the present specification, and within the scope of the invention recited in the claims and equivalents thereof.

What is claimed is:

1. An electronic device comprising:
a memory; and
a processor, wherein the processor executes a program stored in the memory to perform operations comprising:
providing privilege information relevant to a risk requiring attention for a user, on the basis of history information of the user, and wherein the history information of the user is relevant to at least one of date and time, and a location.

2. The electronic device according to claim 1, wherein the processor is further configured to
acquire factor information affecting a health condition of the user, on the basis of the history information of the user, and wherein the history information of the user is relevant to at least one of the date and time and the location, and
provide the privilege information relevant to the risk requiring attention for the user, on the basis of the acquired factor information.

3. The electronic device according to claim 2, wherein the processor is further configured to
calculate an evaluation value indicating an influence of the acquired factor information on the risk, for each type of risk, and
specify the risk requiring attention for the user by determining a level of the risk for each type of risk, on the basis of the calculated evaluation value.

4. The electronic device according to claim 1, wherein the processor is further configured to
acquire information on a behavior of the user, and
provide the privilege information relevant to the risk requiring attention for the user by using the acquired information on the behavior as the history information.

5. The electronic device according to claim 2, wherein the processor is further configured to
specify the risk requiring attention for the user by reflecting respectively influence of the date and time and the location of the factor information on the risk, or influence an elapsed time and a location of the factor information on the risk.

6. The electronic device according to claim 2, wherein the processor is further configured to
acquire information on a behavior of the user, and
specify the risk requiring attention for the user by using the acquired information on the behavior as the factor information.

7. The electronic device according to claim 5, wherein the processor is further configured to
acquire information on a behavior of the user, and
specify the risk requiring attention for the user by reflecting each influence of at least two of the date and time and the location, and the information on the behavior of the factor information on the risk, by using the acquired information on the behavior as the factor information.

8. The electronic device according to claim 2, wherein the factor information includes factor information depending on an area in which the user stays.

9. The electronic device according to claim 2, wherein the factor information includes factor information depending on an environment around the user.

10. The electronic device according to claim 1, wherein the processor is further configured to
provide the privilege information including advice information based on the history information, with respect to the risk requiring attention for the user.

11. The electronic device according to claim 2, wherein the processor is further configured to
determine whether or not the history information of the user is appropriate for a specific condition determined as suitable for health on the basis of the acquired factor information
provide the privilege information for applying a benefit relevant to an insurance to the user in a case where it is determined that the history information of the user is appropriate for the specific condition.

12. A server of a privilege information providing system including an electronic device, and the server configured to be capable of communicating with the electronic device through a network, the server comprising:
a privilege information acquisition section acquiring privilege information relevant to a risk requiring attention for a user, on the basis of history information of the user, and wherein the history information of the user is relevant to at least one of date and time and a location, from the electronic device; and
a privilege information transmission section transmitting the privilege information acquired by the privilege information acquisition section, to the electronic device.

13. A privilege information providing method executed by an electronic device including a processor, the privilege information providing method causing the processor to execute a program stored in a memory to perform operations comprising:
providing privilege information relevant to a risk requiring attention for a user, on the basis of history information of the user, and wherein the history information of the user is relevant to at least one of date and time, and a location.

14. The privilege information providing method according to claim 13, wherein the processor is further configured to
acquire factor information affecting a health condition of the user, on the basis of the history information of the user, and wherein the history information of the user is relevant to at least one of the date and time and the location, and
provide the privilege information relevant to the risk requiring attention for the user, on the basis of the acquired factor information.

15. The privilege information providing method according to claim 14, wherein the processor is further configured to
calculate an evaluation value indicating an influence of the acquired factor information on the risk, for each type of risk, and
specify the risk requiring attention for the user by determining a level of the risk for each type of risk, on the basis of the calculated evaluation value.

16. The privilege information providing method according to claim 13, wherein the processor is further configured to
acquire information on a behavior of the user, and
provide the privilege information relevant to the risk requiring attention for the user by further using the acquired information on the behavior as the history information.

17. The privilege information providing method according to claim 14, wherein the processor is further configured to . . .
specify the risk requiring attention for the user by reflecting respectively influence of the date and time and the location of the factor information on the risk, or influence of an elapsed time and a location of the factor information on the risk.

18. The privilege information providing method according to claim 14, wherein the processor is further configured to
acquire information on a behavior of the user, and
specify the risk requiring attention for the user by further using the acquired information on the behavior as the factor information.

19. The privilege information providing method according to claim 17, wherein the processor is further configured to
acquire information on a behavior of the user, and
specify the risk requiring attention for the user by reflecting each influence of at least two of the date and time and the location, and the information on the behavior of the factor information on the risk, by further using the acquired information on the behavior as the factor information.

20. A non-transitory computer-readable storage medium storing a program that is executed by a computer that comprises a processor to control an electronic device, the program being executable to cause the computer to perform operations comprising:
providing privilege information relevant to a risk requiring attention for a user, on the basis of history information of the user, and wherein the history information is relevant to at least one of date and time, and a location.
